(19) United States
(12) Patent Application Publication
(10) Pub. No.: US 2016/0257990 A1
(43) Pub. Date: Sep. 8, 2016

(54) METHOD AND DEVICE FOR DETECTING MOLECULES OR PARTICLES USING FRACTIONALIZED VOLUMES

(71) Applicant: THE REGENTS OF THE UNIVERSITY OF CALIFORNIA, Oakland, CA (US)

(72) Inventors: Dino Di Carlo, Los Angeles, CA (US); Janay Kong, Los Angeles, CA (US)

(73) Assignee: THE REGENTS OF THE UNIVERSITY OF CALIFORNIA, Oakland, CA (US)

(21) Appl. No.: 15/029,231

(22) PCT Filed: Oct. 17, 2014
(86) PCT No.: PCT/US14/61174

§ 371 Date: Apr. 13, 2016

Related U.S. Application Data
(60) Provisional application No. 61/892,832, filed on Oct. 18, 2013.

(51) Int. Cl. C12Q 1/68

(52) U.S. Cl. CPC C12Q 1/6804; C12Q 1/6844

(57) ABSTRACT

A method of detecting a target and quantifying the concentration of the same within a sample includes generating a plurality of fractionated volumes, wherein at least some of the fractionated volumes contain the target bound to a first detector molecule connected to a first reaction component (R1) and a second detector molecule connected to a second reaction component (R2). The fractionated volumes that contain the target, first detector molecule, second detector molecule, and a probe or other reporter molecule emit light and are imaged. Fractionated volumes emitting radiation can be used to detect the presence of the target within the sample. The number of fractionated volumes emitting a positive emission signal can be counted from the image and the concentration (or range of calculations) of the target can be calculated based at least in part on the number of fractionated volumes emitting a positive emission signal from the image.

### RELATED APPLICATION

This application claims priority to U.S. Provisional Patent Application No. 61/892,932 filed on Oct. 18, 2013, which is hereby incorporated by reference in its entirety. Priority is claimed pursuant to 35 U.S.C. §119.

### STATEMENT REGARDING FEDERALLY SPONSORED RESEARCH AND DEVELOPMENT

This invention was made with Government support under award no. 1332275 awarded by the National Science Foundation (EDISON). The Government has certain rights in the invention.

### FIELD OF THE INVENTION

The technical field generally relates methods and devices used detect the concentration and/or presence of molecules or particles within a sample. More particularly, the invention relates to devices and/or methods in which molecules or particles can be detected with high sensitivity using a modified sandwich enzyme-linked immunosorbent assay (ELISA) that does not require immobilization on a surface or a solid phase.

### BACKGROUND

The typical sandwich ELISA assay utilizes adsorbed antibodies on a surface that capture antigens or targets and a secondary antibody to provide a detection signal. Others have used beads coupled with femtoliter-sized wells to fractionate the sample and potentially generate a higher sensitivity assay. See Patent Application Publication Nos. 2012/0289428, 2012/0196774). However, since this assay requires one bead per each well, the bead capture efficiency limits the sensitivity of the assay. Additionally, both traditional and digital ELISAs require a number of wash steps to remove unbound reagents as well as sequential addition of reagents for binding. Another protein/molecule assay that has been developed is the proximity ligation assay. See Patent Application Publication Nos. 2008/0090238, 2008/0293051). This assay uses two DNA aptamers or antibodies to detect a protein of interest. These detection probes are functionalized with DNA extension molecules, such that when placed in close proximity of each other, are ligated together. This DNA is then amplified and measured with gel electrophoresis or with fluorescent targeted DNA probes. This technique measures the amount of bulk precipitant generated by the presence of the target analyte.

Existing assays require detection antibodies to be immobilized on a surface, including bead-based assays. The assay contemplated herein does not need to be immobilized on a surface for sensitive molecule detection, thus saving cost of materials. Additionally, bead-based assays using fractionated sample volumes to generate a binary on/off signal have lower sensitivity because they require a 1:1 ratio of beads to wells. Thus, in contrast to bead or surface-based assays, the assay contemplated herein does not require a surface or beads and this leads to a higher sensitivity and lower limit of detection. This assay does not require a number of wash steps or sequential addition of reagents, which is typical of current assays. Instead this assay just requires one mix step and then fractionalization. Fractionalization prevents the formation of a false positive signal from components that would otherwise interact in a bulk reaction. While the proximity ligation assay is able to detect proteins with high sensitivity, the bulk measurement technique is more susceptible to noise. Often this technique utilizes a real-time PCR technique that requires multiple thermocycles to achieve a low limit of detection. The gel electrophoresis technique of measuring the amplified DNA requires additional equipment and is costly.

### SUMMARY

The invention relates to devices and/or methods in which molecules or particles can be detected with high sensitivity using a modified sandwich enzyme-linked immunosorbent assay (ELISA) that does not require immobilization on a surface or a solid phase. Using fractionated or segmented volumes, detection signals can be measured as a binary on/off signal, leading to higher sensitivity. This process is based on the premise that both reaction components (that form the sandwich) need to be in the same fractionated sample volume in order for the reaction to proceed. The affinity of the target molecule or particle to each reaction component brings both components together to initiate a measurable reaction. This method can be used as a clinical diagnostic, including in point of care devices. This technique can also be used in analytical research tools.

In one aspect of the invention, a method of quantifying the concentration or range of concentration of a target within a sample includes generating a plurality of fractionated volumes, wherein at least some of the fractionated volumes contain the target bound to a first detector molecule connected to a first reaction component (R1) via a linker molecule, the target also bound to a second detector molecule connected to a second reaction component (R2) via a linker molecule. A reaction is initiated between the first reaction component (R1) and the second reaction component (R2) within the fractionated volumes. The reaction may occur naturally with the passage of time or an external stimulus such as the application of heat (e.g., increased temperature) or light be needed in some embodiments. The plurality of fractionated volumes is then imaged with an imaging device. Typically, a fluorescent probe or other marker is used to identify those fractionated volumes having the target and the reaction components (R1 and R2). The number of fractionated volumes emitting a positive emission signal from the image can be counted using image processing techniques. The concentration or a range of concentrations of the target can then be calculated based at least in part on the number of fractionated volumes emitting a positive emission signal from the image. The number concentration of an initial solution can be estimated by counting the number of volumes with positive emission signals divided by the sample volume that is introduced. Over a concentration range, the concentration is linearly correlated with number of volumes with positive emission signals and calibration with known concentrations can be used to determine the linear coefficient. The molar concentration can be calculated from this number concentration by dividing by Avogadro's number (6.022×10²³) and converting volume to units of liters. At higher concentrations, a non-continuous gradation in positive emission signal in fractionated volumes can also be used to estimate concentration.

In another embodiment, a method, of detecting a target within a sample includes generating a plurality of fractionated volumes, wherein at least some of the fractionated volumes contain the target bound to a first detector molecule connected to a first reaction component (R1), the target also bound to a second detector molecule connected to a second reaction component (R2). A reaction is initiated between the first reaction component (R1) and the second reaction component (R2) within the fractionated volumes. The plurality of fractionated volumes is then imaged using an imaging device. The fractionated volumes emitting a positive emission signal from the image are then identified using image processing techniques.

In another embodiment, a microfluidic device includes an optically transparent substrate and a flexible layer containing a plurality of wells therein, wherein an opening to the plurality of wells faces the substrate and wherein at least one of the flexible layer or the optically transparent substrate is moveable to selectively seal the plurality of wells from each other.

### BRIEF DESCRIPTION OF THE DRAWINGS

FIG. 1 illustrates a target molecule bound to two detector molecules. The two detector molecules are coupled, respectively, to a first reaction component and a second reaction component.

FIG. 2 illustrates an alternative embodiment wherein a linker molecule with multiple reaction components is used to speed up the signal amplification reaction.

FIG. 3A illustrates one type of device used to generate the fractional volumes. The device is a droplet-based device whereby droplets are formed containing the assay reaction components and then are collected in a chamber downstream of the droplet formation region. The droplets accumulate within the downstream chamber in a two-dimensional array. The array of droplets within the chamber may then be imaged.

FIG. 3B illustrates a magnified view of a portion of the droplet-based device of FIG. 3A. Illustrated is the intersection of the reaction components with sheath flow inlets that is used to generate the droplets as well as the downstream chamber where the droplets are collected. The two-dimensional array of droplets is illustrated.

FIG. 4A illustrates another type of device that is used to generate fractional volumes. This embodiment of the device is a two-layer, compression-based device. An inner volume is formed between the two layers and, when brought together in a compression process, forms a plurality of discrete, fractionated volumes. The fractionated volumes can be formed in an array which may then be imaged.

FIG. 4B illustrates a magnified view of a portion of the compression-based device of FIG. 4A. An inlet channel is illustrated that leads the larger region containing a plurality of compartmentalized microwells. Each microwell contains a discrete, fractionated volume in response to compression of the multi-layered device. In FIG. 4B, several of the microwells indicate a “positive” signal that is generated in response to the presence of first and second reaction components (R1, R2) bound to a target molecule.

FIG. 5A illustrates a side, cross-sectional view of the device of FIGS. 4A and 4B in an uncompressed state. The device includes two layers: a PDMS layer that contains the microwells therein and a glass slide that is brought into facing contact with the PDMS layer.

FIG. 5B illustrates a side, cross-sectional view of the device of FIGS. 4A and 4B in a compressed state. The device is interposed between two compression plates that are squeezed together to compress the glass slide against the PDMS layer to form the separate microwells.

FIG. 5C illustrates an alternative embodiment of a compression device.

FIG. 6 illustrates an imaging device used in connection with the microfluidic device according to one embodiment.

FIG. 7A illustrates imaged microwells of the device of FIGS. 4A and 4B. Fluorescent dye (fluorescein) is contained in the microwells which are then imaged with an imaging device.

FIG. 7B illustrates a magnified view of FIG. 7A.

FIG. 8 illustrate DNA electrophoresis gel results illustrating that the LAMP amplification reaction for the entire λ DNA as well as the shortened 600 bp and 1000 bp λ, DNA.

FIG. 9 illustrates a panel of images taken of a device of the embodiment of FIGS. 4A and 4B at times zero (0) and sixty minutes (60) for a control (0 DNA), 600 bp λDNA, and 1 k bp λDNA. The λ DNA samples were diluted 100× in mixed with LAMP reagents in 20 μm well devices.

### DETAILED DESCRIPTION OF THE ILLUSTRATED EMBODIMENTS

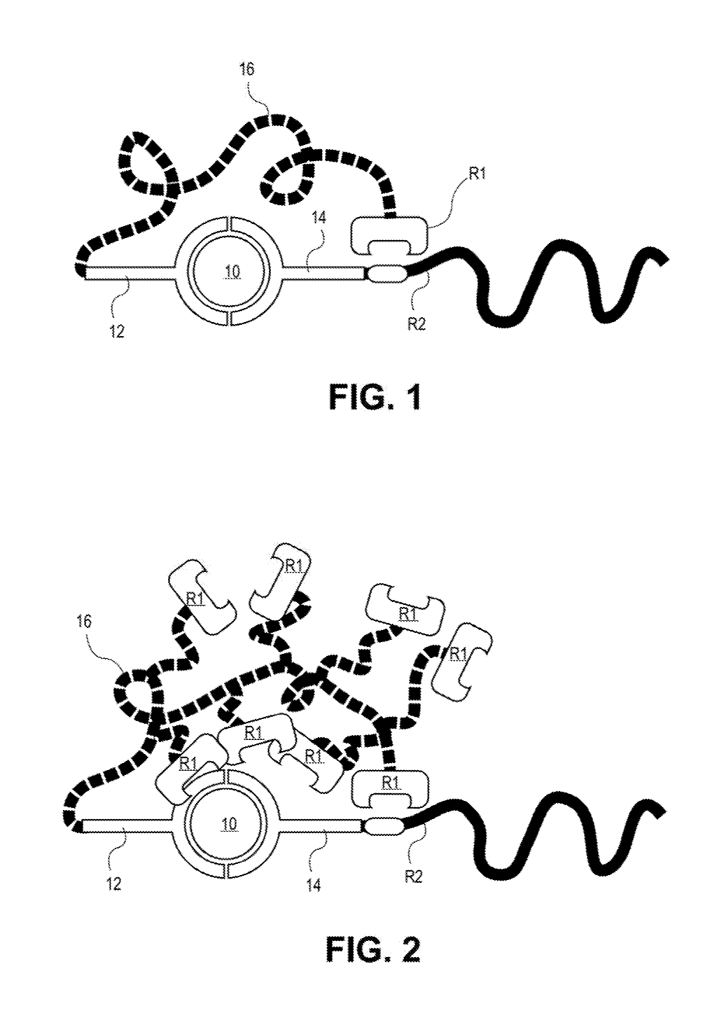

FIG. 1 illustrates the detection mechanism employed in one embodiment. The detection mechanism involves a target 10 which may include a molecule, molecules, or larger particle of interest. The target 10 may include a living particle such as a cell in some embodiments. The detection scheme includes a first detector molecule 12 and a second detector molecule 14. The detector molecules 12, 14 may be the same or different. Typically, the detector molecules 12, 14 are an antibody or aptamer with an affinity to the target 10. For example, the target 10 may include a protein molecule and the first detector molecule 12 and the second detector molecule 14 may include an antibody or aptamer with an affinity to the protein molecule. The target 10 has two or more binding sites that bind to the detector molecules 12, 14. Still referring to FIG. 1, a linker molecule 16 is conjugated to the first detector molecule 12. The linker molecule 16 is typically a long, flexible molecule that connects the first detector molecule 12 to a corresponding reaction component (described in more detail below). The linker molecule 16 may include a polyethylene glycol oligomer, for example. The linker molecule 16 is conjugated at the other end to a first reaction component R1 as illustrated in FIG. 1.

With reference to FIG. 1, a second reaction component R2 is conjugated directly to the second detector molecule 14. Optionally, a linker molecule 16 may be conjugated at one end or region to the second detector molecule 14 and at the other end or region to the second reaction component R2. In this optional embodiment, the linker molecule 16 that connects to the second detector molecule 14 may be the same as, or different from, the linker molecule 16 that is connected to the first detector molecule 12. A key aspect of the linker molecule 16 is that it allows for sufficient motion of a reaction component and is of sufficient length to allow for spatial contact between first and second reaction components (R1, R2) to aid in the progression of the reaction. The linker molecules 16 may be joined with the associated detector molecules 12, 14 and reaction components R1, R2 via any number of bonds including covalent, ionic, electrostatic, and the like.

In one embodiment, the first reaction component R1 is an enzyme, such as DNA polymerase or several DNA polymerases while the second reaction component R2 is a DNA sequence (single strand or multiple strands). For example, R2 may be a single stranded DNA sequence that is used as part of an amplification reaction such as loop-mediated isothermal amplification. The output of the reaction is then converted to an optical signal, for example, by using double-stranded DNA intercalation dye fluorescence (e.g., SYBR® dyes). In another embodiment, the first reaction component R1 may be an enzyme while second reaction component R2 includes a fluorogenic substrate or multiple fluorogenic substrates. In this embodiment, the enzyme (e.g., carboxylesterase) may turnover fluorophores from the fluorogenic substrate(s) (e.g., fluorescein diacetate) to generate a fluorescent signal. Generally, it is preferable to have multiple reaction sites so that the signal may be amplified and detected.

In order to detect a target 10 of interest, the first and second detector molecules 12, 14, such as antibodies, are conjugated directly or indirectly through a linker 16 to first and second reaction components R1, R2. Only when both detector molecules 12, 14 are attached to the target analyte 10 will a “sandwich” be formed, bringing the first reaction component R1 and the second reaction component R2 together as shown in FIG. 1. The presence of the target 10 and the bound detector molecules 12, 14 brings both reaction components R1, R2 into the same fractionated volume 18, initiating the reaction and generating a positive signal. Alternatively, temperature adjustment or the application of light can be used following fractionation to initiate the reaction (e.g. a temperature increase for loop-mediated isothermal amplification). This approach prevents pre-mature reaction of R1 and R2 prior to fractionation which would lead to a higher background signal. Fractionated volume 18 refers to a geographically discrete area of a testing device 10, 40. The reaction that generates a positive signal may include, in one embodiment, nucleic acid amplification. In another embodiment, the reaction may include, for example, an enzyme-mediated reaction to form fluorescent compounds from fluorogenic substrates or species. For some reactions, such as nucleic acid amplification, a dye is used to generate an optical signal by intercalation with the nucleic acid that then fluoresces in response to illumination with excitation light. In some embodiments, where fluorogenic substrates are used, no such dye is needed. Without the presence of the target 10, the levels of both detector molecules 12, 14 are low enough that the probability of being in the same fractionated sample volume is also low, leading to a low and predictable number of fractionated or segregated areas with a background positive signal.

FIG. 2 illustrates an alternative detection configuration whereby a linker 16 is connected with multiple first reaction components R1. The multiple reaction sites allow for quicker reaction progression to achieve sufficient signal for fluorescence or colorimetric detection in a shorter time period. As seen in FIG. 2, there are eight (8) copies of reaction component R1. The linker 16 may be a branched structure to aid in providing multiple reaction sites. While FIG. 2 illustrates multiple first reaction components R1 it should be understood that multiple second reaction components R2 could also be provided.

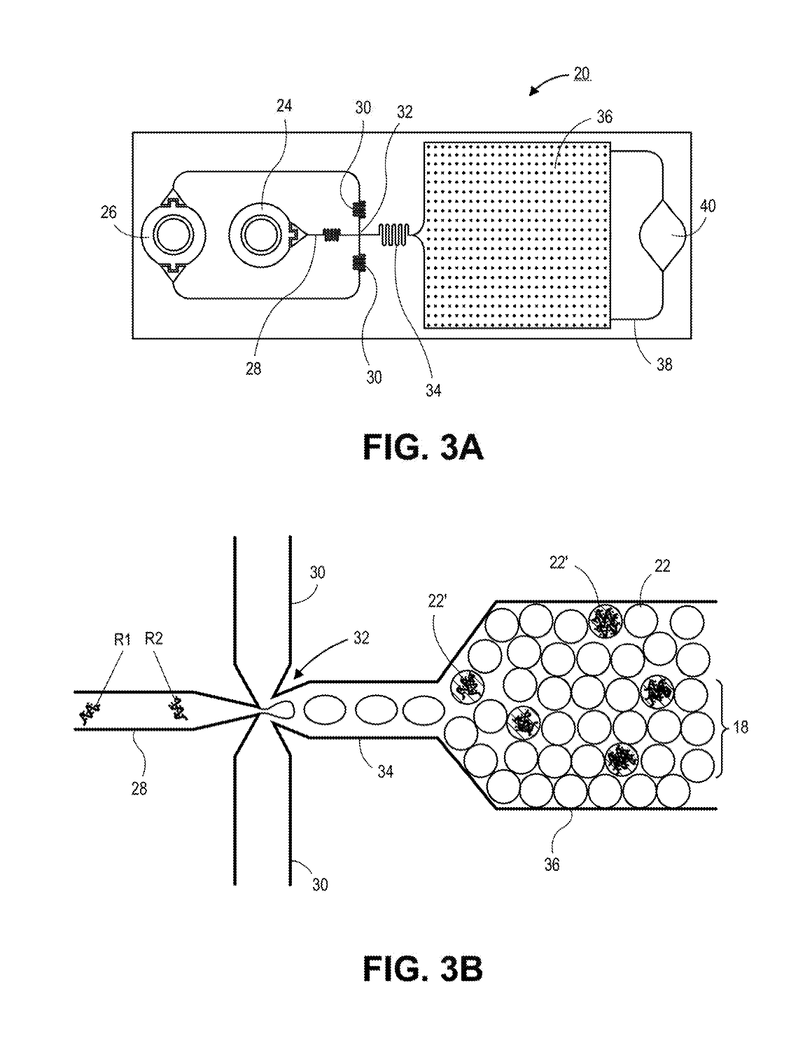

FIG. 3A illustrates one type of device 20 used to generate the fractional volumes 18 (illustrated in FIG. 3B). In this embodiment, the device 20 is a microfluidic device that generates micro-emulsions (i.e., droplets 22) that act as the fractional volumes 18. The droplets may have a variety of diameters but are typically greater than 10 μm. The device 20 includes a first inlet 24 that is used to deliver a sample with the target 10 as well as the reaction mixture that contains the first detector molecule 12 (and associated first reaction component R1) and the second detector molecule 14 (and the second reaction component R2). Typically, this fluid is an aqueous fluid. A second inlet 26 is provided that can be used to deliver a fluid that is immiscible with the fluid delivered via the first inlet 24. For example, an oil-based fluid may be delivered to the second inlet 26. Fluid may be delivered to the first inlet 24 and second inlet 26 via respective fluid pumps (e.g., syringe pumps or the like; not shown). A channel 28 coupled to the first inlet 24 and intersects with two opposing channels 30 connected to the second inlet 26. The intersection of the channels 28, 30 forms a droplet generation region 32 whereby the aqueous droplets 22 are pinched off using a sheath flow from the opposing channels 30. Another channel 34 connects the droplet generation region 32 to a downstream droplet collection region 36. The droplet collection region 36 is a three dimensional chamber with a large width and length but small height so that droplets 22 form a two-dimensional array within the droplet collection region 36. The droplet collection region 36 is constructed with a height so that the droplets 22 do not stack in multiple layers which would interfere with the imaging of the discrete fractional volumes 18 which is needed for detection and concentration determination. The droplet collection region 36 is coupled via channel 38 to an outlet 40 whereby the droplets 22 fluids and like can be removed from the device 20.

FIG. 3B illustrates a magnified view of the droplet generation region 32 and the downstream droplet collection region 36. Reaction components including the detector molecules 12, 14 and their respective reaction components R1, R2 are illustrated within channel 28. Droplets are pinched off in the droplet generation region 32 and collected in the droplet collection region 36. Certain droplets 22′ contained within the droplet generation region 32 exhibit a positive optical signal because they contain the target 10 and detector molecules 12, 14 (with associated reaction components R1, R2). The optical signal is binary in that each droplet either emits a positive optical signal or not in this case. The invention requires that reactions are compartmentalized such that reaction components R1 and R2 have less probability to interact in a random fashion but react more commonly when bound to the target 10 and therefore can be present within the same fractional volume 18 at higher levels than Poisson statistics would dictate. Fractionalization also allows for a diffusible signal to accumulate to higher levels that can be more easily interrogated—another critical component of the invention.

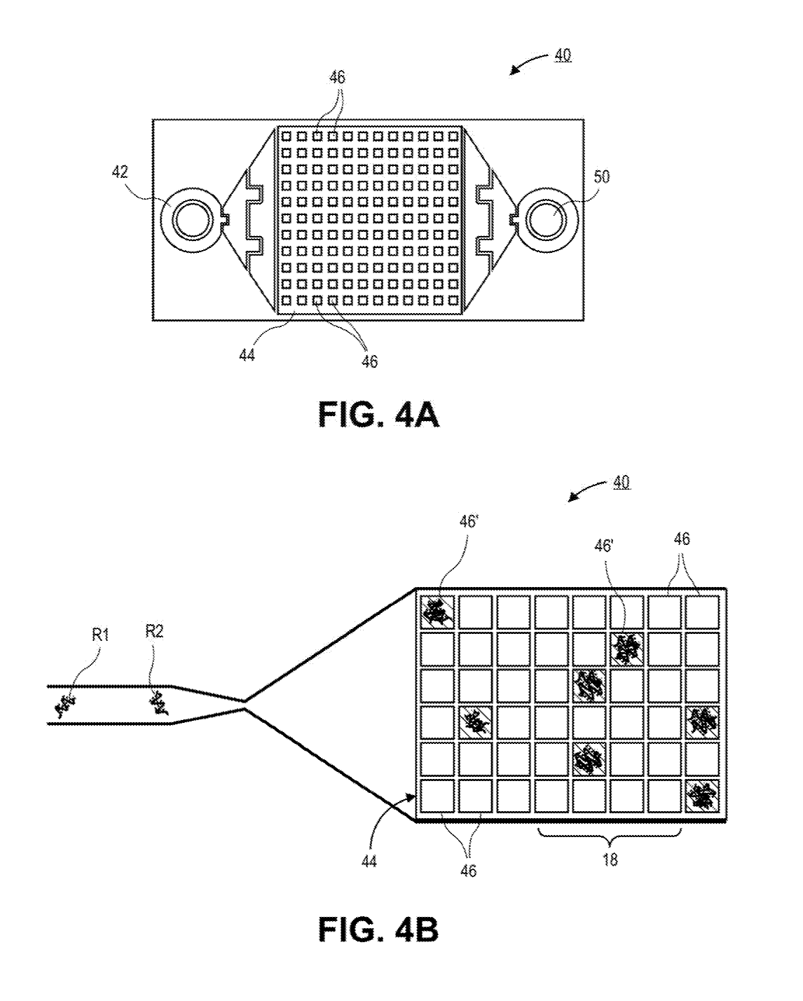

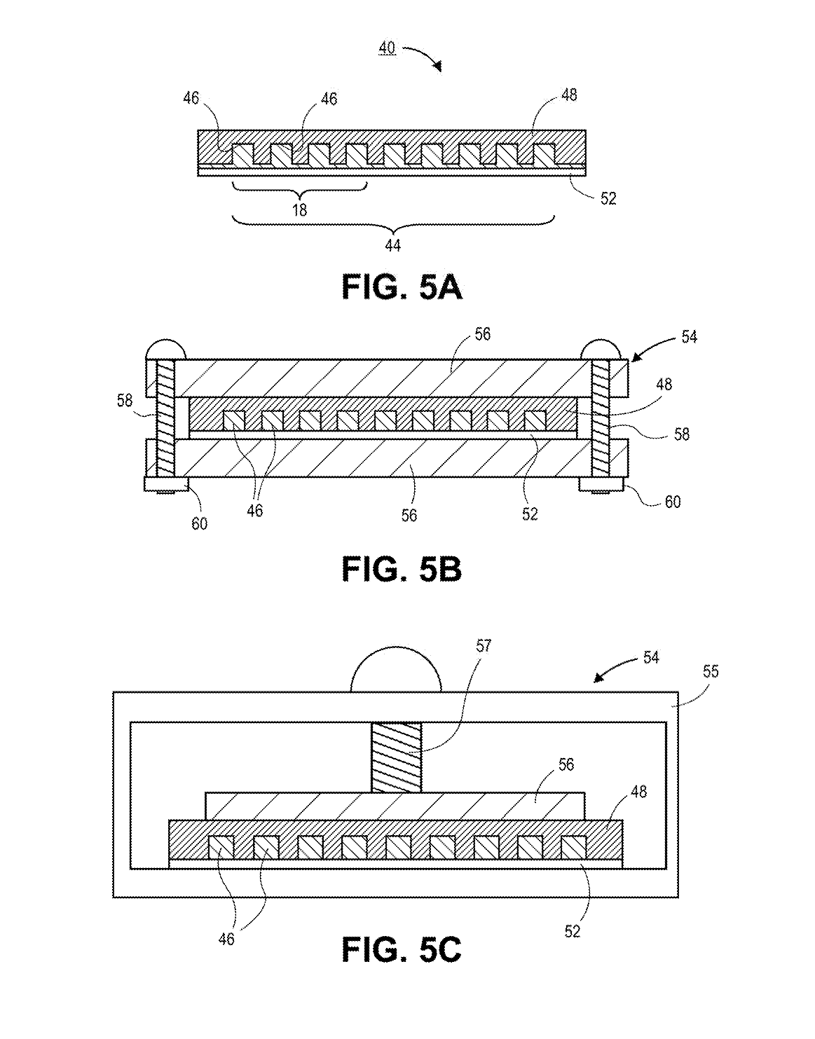

FIG. 4A illustrates another embodiment of a device 40. In this embodiment, the fractional volumes 18 (as seen in FIG. 4B) are created using a plurality of discrete microwells 46 which are formed in the device 40. Some of the microwells 46′ contain fractional volumes 18 that are positive in that first and second reaction components R1, R2 bind to a target 10 and emit a positive optical signal. With reference to FIG. 4A, the device 40 includes an inlet 42 that is fluidically connected to a region 44 holding a plurality of microwells 46. The microwells 46 are formed in a flexible layer 48 (FIGS. 5A-5C) which may include a flexible substrate such as polydimethylsiloxane (PDMS). Each microwell 46 forms a fractional volume 18 such that when the device 40 is fully assembled and loaded with fluid forms a discrete, separate reaction volume. The size of the microwells 46 may vary but microwells 46 having a width of around 15 μm and height greater than or equal to 60 μm are preferred. The larger the total volume of confined regions the lower the detection limit for the assay. The device 40 further includes an outlet 50 whereby fluids can be removed from the device 40. With reference to FIGS. 5A and 5B, the device 40 is a two-layer device that is formed from by compressing the flexible layer 48 containing the microwells 46 against an optically transparent substrate 52. The optically transparent substrate 52 may include a glass slide although other optically transparent materials may be used (e.g., plastics, etc.). During use, the two-layer device 40 is filled with the mixed assay components that include the target 10 and detector molecules 12, 14 (with associate reaction components R1, R2) until all the microwells 46 are filled and all dead volume is gone. There exists a small height in all areas outside the microwells 46 to ensure complete fractionalization once the device 40 is compressed.

FIG. 5A illustrates a side view of the device 40 in an uncompressed state. A thin layer of fluid is seen interposed between the upper surface of the microwells 46 and the opposing optically transparent substrate 52. There exists a gap or height in this state whereby fluid containing the mixed assay components can be introduced into the microwells 46. The fluid may be delivered to the device via the inlet 42 and removed from the device from the outlet 50. In one embodiment, only the inlet 42 is needed given that dead volume gas is able to exit the device via the flexible layer 48 (e.g., gas permeable PDMS layer).

With reference to FIG. 5B, a compression device 54 is used to compress the flexible layer 48 and the optically transparent substrate 52 together. The compression device 54 in FIG. 5B includes compression plates 56 that press against the flexible layer 48 and the optically transparent substrate 52. Threaded screws 58 passing through the compression plates 56 have nuts 60 that can be tightened to compress the flexible layer 48 and the optically transparent substrate 52. FIG. 5C illustrates an alternative embodiment of a compression device 54 that uses a housing 55 with a single compression plate 56 that is operated by a screw 57. Note that in the embodiment of FIG. 5B, the housing 55 should still provide access to view and image the region 44 containing the microwells 46. Thus, the housing 55 (or compression plates 56 for the embodiment of FIG. 5B) may press on regions of the flexible layer 48 and the optically transparent substrate 52 that do not overlie the region 44. Alternatively, the compression plates 56 may be optically transparent themselves. FIG. 5B illustrate the device 40 in the compressed state whereby there no longer is the thin layer of fluid between the flexible layer 48 and the optically transparent substrate 52 together. Instead, the fluid is trapped within the microwells 46 that have become discrete fractional volumes 18 that are separated from one another.

The devices 20, 40 may be patterned using photolithography, and then prototyped using PDMS or other materials used for microfluidic devices. Alternatively, this can also be made using epoxy or using an injection molding process. In the embodiment that uses device 40, the flexible layer 48 needs to have some degree of flexibility to form the fractional volumes 18 during the compression operation.

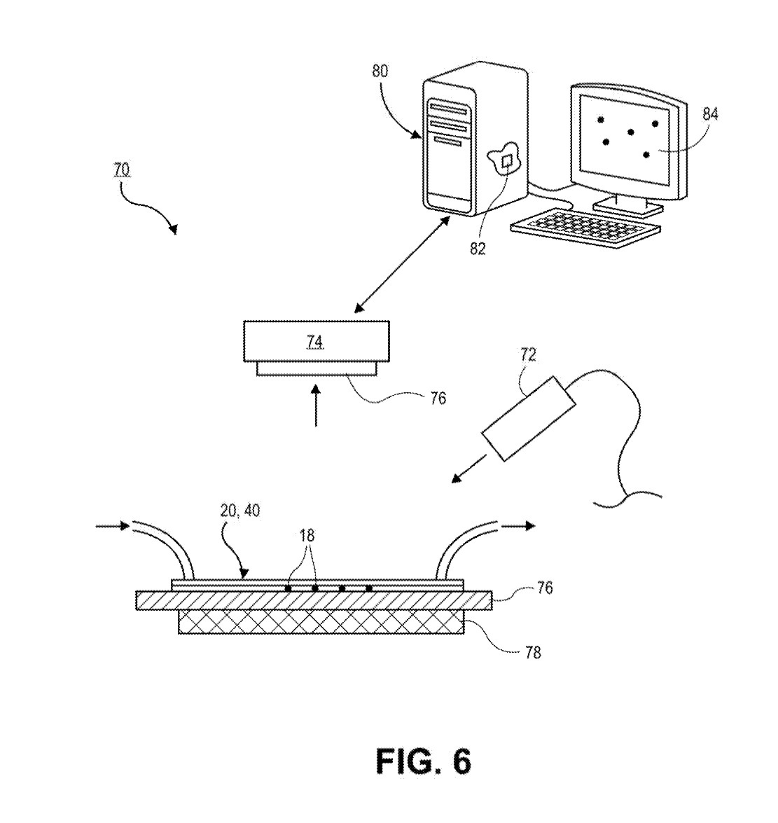

During use, a sample that is known or believed to contain the target 10 is mixed with metered concentrations of both reaction component assemblies (i.e., the detector molecules 12, 14 with associated reaction components R1, R2 in stoichiometric excess required for reaction and then flowed through the device 20, 40 to create fractionated volumes 18. The reaction is then proceeds or is initiated (e.g., by passage of time, change in temperature, exposure to light, etc.), and an optical response is measured using an imaging device 70 as illustrated in FIG. 6.

FIG. 6 illustrates an imaging device 70 according to one embodiment. The imaging device 70 includes an excitation light source 72 that is used to illuminate the region of the device 20, 40 that contains the fractional volumes 18. The excitation light source 72 may include an emission filter as is known in the art so that the device 20, 40 is illuminated at the wavelength range that causes the fluorescent probe or dye to fluoresce. The imaging device 70 includes an imager 74 that captures images of the region of the device 20, 40 that contains the fractional volumes 18. The imager 74 may include a microscope with an attached camera or it may include a solid state imaging device such as a CCD or CMOS image sensor. The imager 74 may also be a small portable imaging device that may be used in connection with a mobile communication device such as a mobile phone. An example of such a system is disclosed in U.S. Patent Application Publication No. 2012/0157160 which is incorporated by reference as if set forth fully herein. A filter 76 may be used so that only fluorescent light is being imaged by the imager 74. As seen in FIG. 6, the device 20, 40 is held atop a support 76. FIG. 6 also illustrates a heater 78 that, in some embodiments, may be needed to heat the fractional volumes 18 to promote or accelerate reaction. The heater 78 may include a thermoresistive heater or other similar device. The heater 78 may be controlled through a controller (not shown) or through computer 80.

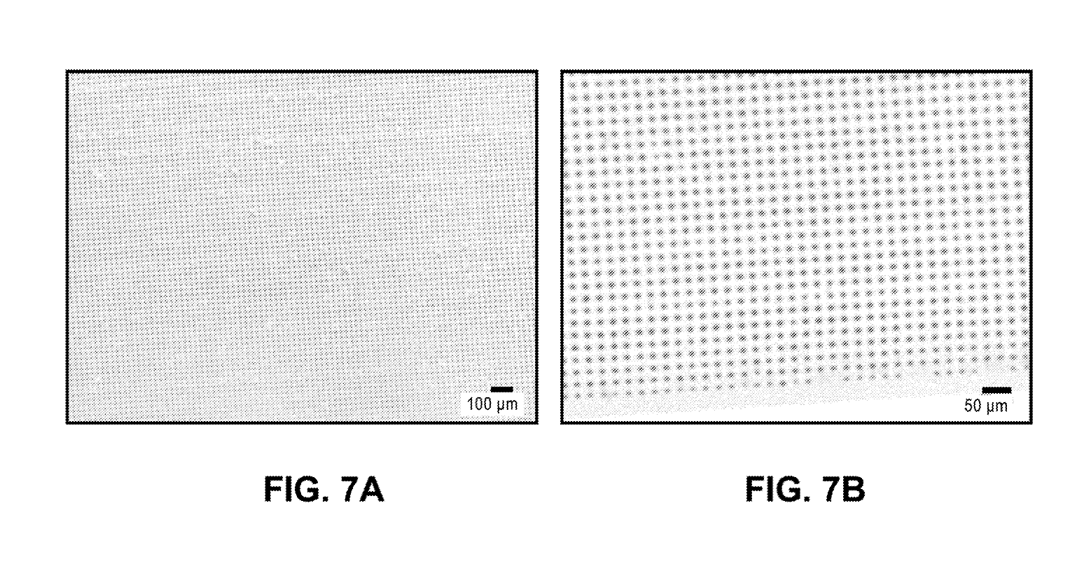

The computer 80 in FIG. 6 contains at least one processor 82 therein and runs imaging software. Image frames obtained from the imager 74 can be transferred or otherwise accessed by the computer 80 for image processing. The computer 80 may include a display 84 that is used to display, for example, the 2D array of fractional volumes 18. The display 84 may illustrate the positive fractional volumes 18 that are detected in the device 20, 40. The display 84 may also display a concentration or concentration range that is based in part on the number of positive fractional volumes 18 that are observed in the device 20, 40. Image software such as ImageJ software that is executed by the processor 82 using computer 80 may be used to detect and quantify fluorescently labeled fractional volumes 18. The software may perform background subtractions and contrast enhancement. Filtering can be applied to filter out unwanted spatial frequencies in intensity. This can be used, together with a thresholding or local maxima detection scheme to quantify “positive” results. Namely, a light intensity over a pre-determined threshold value may be characterized as positive. Or over a set of pre-determined values to indicate multiple binding reactions per confined volume in another embodiment. FIGS. 7A and 7B illustrates two images (at different magnifications) of the microwells 46 of the device 40 of FIGS. 4A and 4B. Fluorescent dye (fluorescein) is contained in the microwells 46 which are then imaged with an imaging device 70.

With the optimal concentration of detector molecules 12, 14, Poisson statistics allow for the calculation of the concentration of the target 10 and number of background fractionated volumes 18 that are likely to fluoresce because R1 and R2 are present in a confined volume by chance. Note that this fluorescent level may be lower than when R1 and R2 are joined by the target 10 such that they are in close proximity to react more rapidly. Tables 1 and 2 below illustrate such calculations, with potential concentrations of interest highlighted (Reaction Component Probability is a proxy for the concentration of the reactants). With very low reaction component concentrations, noise can be minimized (false positive signals generated by fractionated volumes containing reaction component R1 and reaction component R1 without any target present) to allow for extremely low limits of detection. However, with such low concentrations, the calculation of the concentration of the target 10 will have a limited range of possible values. With higher reaction component concentrations, the noise level will be higher, but calculation of the target 10 Poisson statistics will be more accurate for higher concentration range measurements where R1 and R1 are no longer limiting reagents for detecting target 10. This device 20, 40 can be adapted to use whichever concentration level is necessary depending on assay requirements, or potentially, use multiple concentrations in a parallel assay.

TABLE 1
Oil/Water Droplet Fractionation Probability Statistics

| Reaction | | | | Droplet | Droplet | | |
|---|---|---|---|---|---|---|---|
| Component | No. of | | Max | Diameter | Volume | Conc Low | Conc High |
| Probability | Droplets | Noise | Signal | (μm) | (L) | (M) | (M) |
| 0.001 | 1000000 | 1 | 1000 | 10 | 5.23599E−13 | 1.15012E−16 | 1.15012E−13 |
| 0.01 | 1000000 | 100 | 10000 | 10 | 5.23599E−13 | 1.15012E−14 | 1.15012E−12 |
| 0.001 | 1000000 | 1 | 1000 | 5 | 6.54498E−14 | 9.20094E−16 | 9.20094E−13 |
| 0.01 | 1000000 | 100 | 10000 | 5 | 6.54498E−14 | 9.20094E−14 | 9.20094E−12 |
| 0.001 | 10000000 | 10 | 10000 | 10 | 5.23599E−13 | 1.15012E−16 | 1.15012E−13 |
| 0.01 | 10000000 | 1000 | 100000 | 10 | 5.23599E−13 | 1.15012E−14 | 1.15012E−12 |
| 0.001 | 10000000 | 10 | 10000 | 5 | 6.54498E−14 | 9.20094E−16 | 9.20094E−13 |
| 0.01 | 10000000 | 1000 | 100000 | 5 | 6.54498E−14 | 9.20094E−14 | 9.20094E−12 |
| 0.001 | 100000 | 0.1 | 100 | 10 | 5.23599E−13 | 1.15012E−16 | 1.15012E−13 |
| 0.01 | 100000 | 10 | 1000 | 10 | 5.23599E−13 | 1.15012E−14 | 1.15012E−12 |
| 0.001 | 100000 | 0.1 | 100 | 5 | 6.54498E−14 | 9.20094E−16 | 9.20094E−13 |
| 0.01 | 100000 | 10 | 1000 | 5 | 6.54498E−14 | 9.20094E−14 | 9.20094E−12 |
| 0.001 | 1000000 | 1 | 1000 | 20 | 4.18879E−12 | 1.43765E−17 | 1.43765E−14 |
| 0.01 | 1000000 | 100 | 10000 | 20 | 4.18879E−12 | 1.43765E−15 | 1.43765E−13 |
| 0.005 | 1000000 | 25 | 5000 | 20 | 4.18879E−12 | 3.59412E−16 | 7.18823E−14 |
| 0.05 | 1000000 | 2500 | 50000 | 20 | 4.18879E−12 | 3.59412E−14 | 7.18823E−13 |
| 0.005 | 1000000 | 25 | 5000 | 5 | 6.54498E−14 | 2.30023E−14 | 4.60047E−12 |
| 0.05 | 1000000 | 2500 | 50000 | 5 | 6.54498E−14 | 2.30023E−12 | 4.60047E−11 |
| 0.005 | 1000000 | 25 | 5000 | 8 | 2.68083E−13 | 5.61581E−15 | 1.12316E−12 |
| 0.05 | 1000000 | 2500 | 50000 | 8 | 2.68083E−13 | 5.61581E−13 | 1.12316E−11 |
| 0.001 | 100000 | 0.1 | 100 | 20 | 4.18879E−12 | 1.43765E−17 | 1.43765E−14 |
| 0.01 | 100000 | 10 | 1000 | 20 | 4.18879E−12 | 1.43765E−15 | 1.43765E−13 |
| 0.001 | 100000 | 0.1 | 100 | 15 | 1.76715E−12 | 3.40775E−17 | 3.40775E−14 |
| 0.01 | 100000 | 10 | 1000 | 15 | 1.76715E−12 | 3.40775E−15 | 3.40775E−13 |
| 0.005 | 100000 | 2.5 | 500 | 20 | 4.18879E−12 | 3.59412E−16 | 7.18823E−14 |
| 0.05 | 100000 | 250 | 5000 | 20 | 4.18879E−12 | 3.59412E−14 | 7.18823E−13 |
| 0.005 | 100000 | 2.5 | 500 | 15 | 1.76715E−12 | 8.51939E−16 | 1.70388E−13 |
| 0.05 | 100000 | 250 | 5000 | 15 | 1.76715E−12 | 8.51939E−14 | 1.70388E−12 |
| 0.005 | 100000 | 2.5 | 500 | 30 | 1.41372E−11 | 1.06492E−16 | 2.12985E−14 |
| 0.05 | 100000 | 250 | 5000 | 30 | 1.41372E−11 | 1.06492E−14 | 2.12985E−13 |
| 0.01 | 100000 | 10 | 1000 | 30 | 1.41372E−11 | 4.25969E−16 | 4.25969E−14 |

TABLE 2
Two-Layer Compression Compartmentalization Probability Statistics

| Reaction | | | | Square | Compartment | | |
|---|---|---|---|---|---|---|---|
| Component | # | | Max | Dimension | Volume | Conc Low | Conc High |
| Probability | Compartments | Noise | Signal | (μm) | (L) | (M) | (M) |
| 0.001 | 160000 | 0.16 | 160 | 15 | 1.35E−11 | 4.46074E−18 | 4.46074E−15 |
| 0.01 | 160000 | 16 | 1600 | 15 | 1.35E−11 | 4.46074E−16 | 4.46074E−14 |
| 0.001 | 110889 | 0.110889 | 110.889 | 20 | 2.4E−11 | 2.50917E−18 | 2.50917E−15 |
| 0.01 | 110889 | 11.0889 | 1108.89 | 20 | 2.4E−11 | 2.50917E−16 | 2.50917E−14 |
| 0.005 | 110889 | 2.772225 | 554.445 | 20 | 2.4E−11 | 6.27292E−17 | 1.25458E−14 |
| 0.05 | 110889 | 277.2225 | 5544.45 | 20 | 2.4E−11 | 6.27292E−15 | 1.25458E−13 |
| 0.005 | 160000 | 4 | 800 | 15 | 1.35E−11 | 1.11519E−16 | 2.23037E−14 |
| 0.05 | 160000 | 400 | 8000 | 15 | 1.35E−11 | 1.11519E−14 | 2.23037E−13 |
| 0.005 | 160000 | 4 | 800 | 15 | 1.35E−11 | 1.11519E−16 | 2.23037E−14 |

The concentration or a range of concentrations of the target 10 can then be calculated based at least in part on the number of fractionated volumes 18 emitting a positive emission signal from the image. The number concentration of an initial solution can be estimated by counting the number of fractionated volumes 18 with positive emission signals divided by the sample volume introduced. Over a concentration range described in Tables 1 and 2, concentration is linearly correlated with number of volumes with positive emission signals and calibration with known concentrations can be used to determine the linear coefficient of correlation. The molar concentration can be calculated from this number concentration by dividing by Avogadro's number (6.022×10²³) and converting volume to units of liters. At higher concentrations, a non-continuous gradation in positive emission signal in fractionated volumes can also be used to estimate concentration as described in Rissin et al., simultaneous detection of single molecules and singulated ensembles of molecules enables immunoassays with broad dynamic range, Anal Chem. 2011 Mar. 15; 83(6):2279-85, which is incorporated by reference as if set forth fully herein.

### Experimental

As noted above, in one embodiment of the invention, first reaction component R1 is an enzyme, such as DNA polymerase or several DNA polymerases while the second reaction component R2 is a DNA sequence (single strand or multiple strands). In this experiment, shortened sequences of λ DNA were tested for their ability to amplify and be detected within microwells of the device of the type illustrated in FIGS. 4A and 4B. The experiment utilized loop-mediated isothermal amplification (LAMP) which is an amplification method that amplifies DNA under isothermal conditions using a set of four specialty designed primers and DNA polymerase. Details regarding the LAMP process may be found in Nagamine et al., Accelerated reaction by loop-mediated isothermal amplification using loop primers, Molecular and Cellular Probes, 16, 223-229 (2002), which is incorporated by reference as if set forth fully herein. The primers used are set forth below.

TABLE 3

| Primer | Sequence (5′ −> 3′) |
|---|---|
| FIP | SEQ ID NO: 1 |
| BIP | SEQ ID NO: 2 |
| F3 | SEQ ID NO: 3 |
| B3 | SEQ ID NO: 4 |
| Loop F | SEQ ID NO: 5 |
| Loop B | SEQ ID NO: 6 |

Two shortened sequences of λ DNA were investigated for amplification within the microwells of the device illustrated in FIGS. XX. The shortened sequences included a 600 bp shortened sequence of λ DNA (SEQ ID NO:7) and a 1 k shortened sequence of λ DNA (SEQ ID NO:8). The LAMP reaction was run with 25 μl of a 2× reaction mix containing 2M betaine, 40 mM Tris-HCL (pH 8.8), 20 mM KCL, 20 mM (NH₄)₂SO₄, 12 mM MgSO₄, 0.2% Triton-X 100, and 3.2 mM dNTPs in combination with DNA, primer mix, SYBR green, DNA polymerase, and water. 5 μl of DNA (5.74×10¹⁰ copies/4) was used with 2 μL of the primer mix. The primer mix included 16 μM FIP, 16 μM BIP, 2 μM F3, 2 μM B3, 4 μM Loop F, and 4 μM Loop B. 2 μl of SYBR® (1000× diluted) was used for the fluorescent dye. The solution included 2 μL of DNA polymerase (BST polymerase, large fragment (8,000 units/mL), and 14 μL ultrapure water (DNAse/RNAse free water).

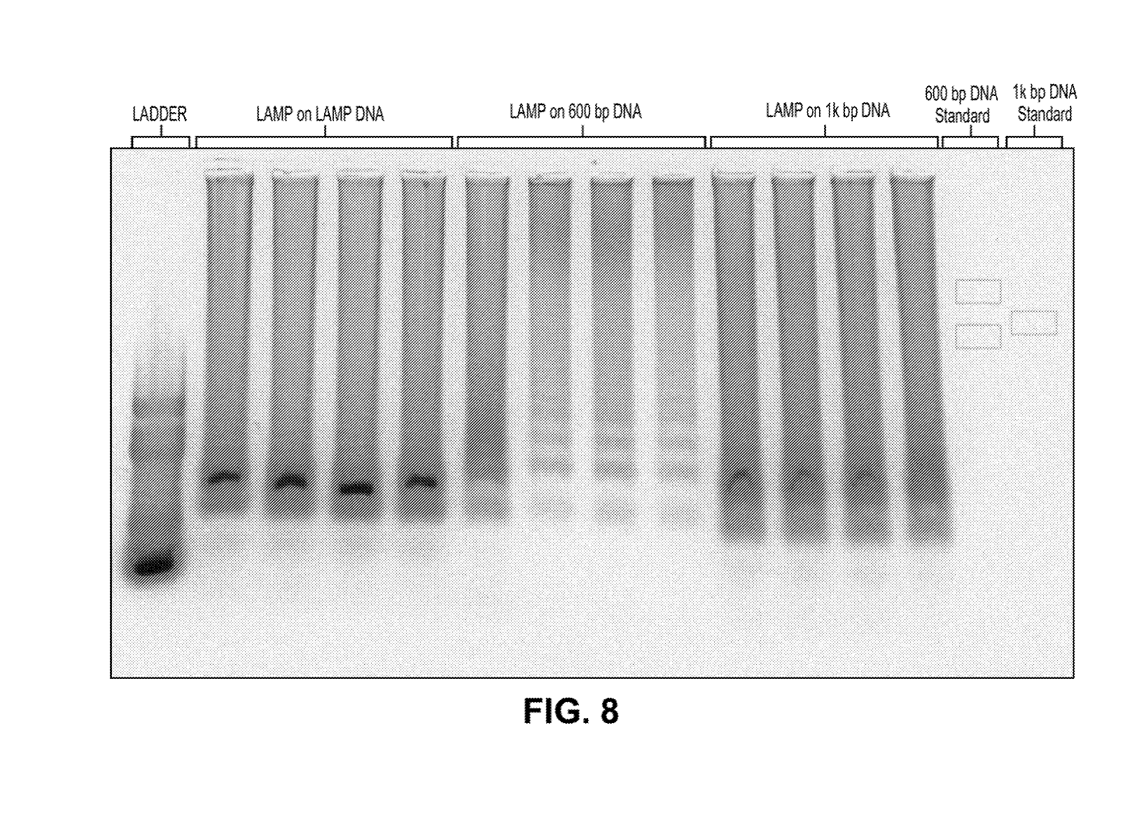

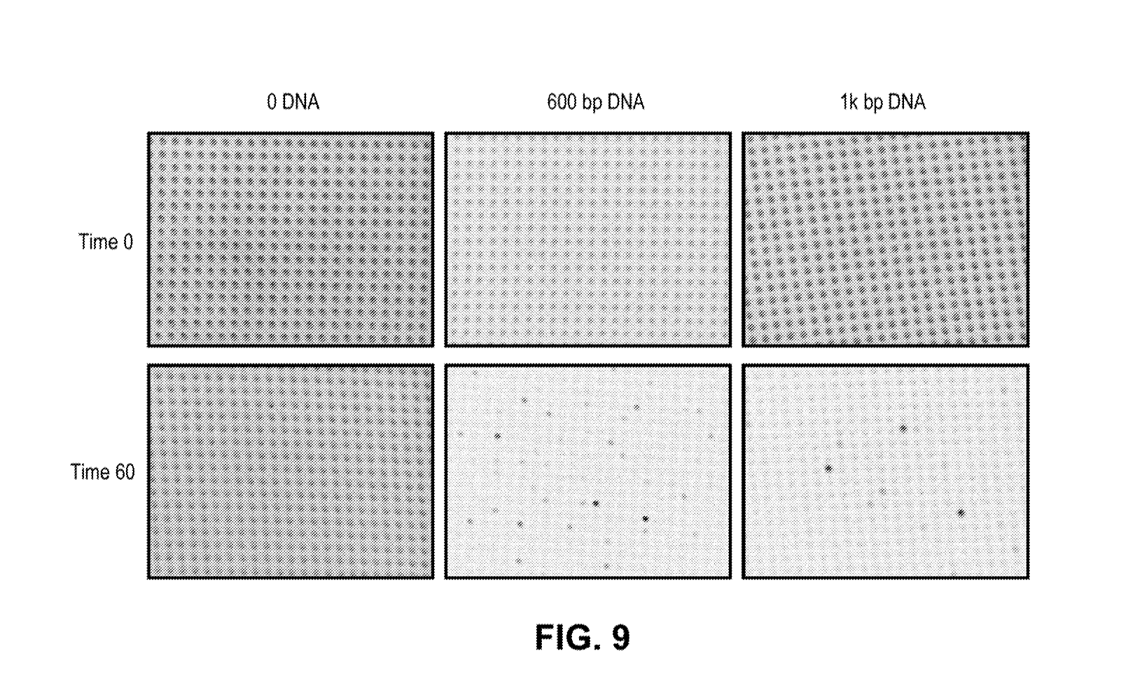

FIG. 8 illustrate DNA electrophoresis gel results illustrating that the LAMP amplification reaction for the entire λ DNA as well as the shortened 600 bp and 1000 bp λ DNA. All LAMP materials were mixed together on ice and incubated at 65° C. for one hour. FIG. 9 illustrates fluorescent images obtained of the device of FIGS. 4A and 4B after the addition of 100× diluted DNA. In this experiment, all of the LAMP reaction components were mixed together on ice and the device was filled and then compressed. The device was incubated at 65° C. for one hour. The device was then imaged with fluorescein isothiocyanate (FITC) filter set to detect SYBR green fluorescence of double stranded DNA. The device has microwells with diameters of 20 μm and height of 60 μm. Both the zero (0) DNA and initial time zero (0) conditions illustrate a low signal to noise ratio (because of the expected low signal). FIG. 9 also illustrates the same microwells after amplification (time=60 minutes). Note that in the 600 bp and the 1 k by experiments, the contrast adjusted microwells show a higher signal to noise ratio (higher signal compared to background noise).

While embodiments of the present invention have been shown and described, various modifications may be made without departing from the scope of the present invention. The invention, therefore, should not be limited, except to the following claims, and their equivalents.

1. A method of quantifying the concentration of a target within a sample comprising:
generating a plurality of fractionated volumes, wherein at least some of the fractionated volumes contain the target bound to a first detector molecule connected to a first reaction component (R1) via a linker molecule, the target also bound to a second detector molecule connected to a second reaction component (R2) via a linker molecule;
imaging the plurality of fractionated volumes;
counting the number of fractionated volumes emitting a positive emission signal from the image; and
calculating the concentration or range of concentrations of the target based at least in part on the number of fractionated volumes emitting a positive emission signal from the image.

2. The method of claim 1, wherein the fractionated volumes comprise droplets.

3. The method of claim 1, wherein the fractionated volumes are contained within microwells.

4. The method of claim 1, wherein a plurality of first reaction components (R1) are connected to the first detector molecule via a linker molecule.

5. The method of claim 1, wherein a plurality of second reaction components (R2) are connected to the second detector molecule via a linker molecule.

6. The method of claim 1, wherein the first reaction component (R1) comprises DNA polymerase.

7. The method of claim 6, wherein the second reaction component (R2) comprises a single strand of DNA or multiple strands of DNA.

8. The method of claim 7, wherein the fractionated volumes further comprise a fluorescent marker specific to amplified DNA.

9. The method of claim 1, wherein the calculated concentration is based on a Poisson statistical analysis.

10. The method of claim 1, further comprising initiating a reaction between the first reaction component (R1) and the second reaction component (R2).

11. The method of claim 10, wherein initiating the reaction is accomplished by altering the temperature of the fractionated volumes.

12. The method of claim 10, wherein initiating the reaction is accomplished by illuminating the fractionated volumes with light.

13. The method of claim 10, wherein the plurality of fractionated volumes is imaged after a period of time has elapsed.

14. A method of detecting a target within a sample comprising:
generating a plurality of fractionated volumes, wherein at least some of the fractionated volumes contain the target bound to a first detector molecule connected to a first reaction component (R1), the target also bound to a second detector molecule connected to a second reaction component (R2);
initiating a reaction between the first reaction component (R1) and the second reaction component (R2);
imaging the plurality of fractionated volumes; and
identifying the fractionated volumes emitting a positive emission signal from the image.

15. The method of claim 14, wherein the first detector molecule is connected to the first reaction component (R1) via a linker and the second detector molecule is connected to the second reaction component (R2) via a linker.

16. The method of claim 14, wherein the fractionated volumes comprise droplets.

17. The method of claim 14, wherein the fractionated volumes are contained within microwells.

18. The method of claim 14, wherein the first reaction component (R1) comprises DNA polymerase.

19. The method of claim 18, wherein the second reaction component (R2) comprises a single strand of DNA or multiple strands of DNA.

20. The method of claim 14, wherein the fractionated volumes comprise a fluorescent marker that increases in intensity upon reaction.

21. The method of claim 14, wherein the reaction is initiated by altering the temperature of the fractionated volumes.

22. The method of claim 14, wherein the reaction is initiated by illuminating the fractionated volumes with light.

23-26. (canceled)


SEQUENCE LISTING (8 sequences)

SEQ ID NO: 1 - DNA, 46 bp, Artificial Sequence
  Oligonucleotide primer
cagccagccgcagcacgttcgctcataggagatatggtagagccgc

SEQ ID NO: 2 - DNA, 51 bp, Artificial Sequence
  Oligonucleotide primer
gagagaatttgtaccacctcccaccgggcacatagcagtcctagggacagt

SEQ ID NO: 3 - DNA, 22 bp, Artificial Sequence
  Oligonucleotide primer
ggcttggctctgctaacacgtt

SEQ ID NO: 4 - DNA, 22 bp, Artificial Sequence
  Oligonucleotide primer
ggacgtttgtaatgtccgctcc

SEQ ID NO: 5 - DNA, 17 bp, Artificial Sequence
  Oligonucleotide primer
ctgcatacgacgtgtct

SEQ ID NO: 6 - DNA, 20 bp, Artificial Sequence
  Oligonucleotide primer
accatctatgactgtacgcc

SEQ ID NO: 7 - DNA, 600 bp, Coliphage lambda
tgcatgctagatgctgatatattttagaggtgataaaattaactgcttaactgtcaatgtaatacaagttgtttgatctttgcaatgattcttatcagaaaccatatagtaaattagttacacaggaaatttttaatattattattatcattcattatgtattaaaattagagttgtggcttggctctgctaacacgttgctcataggagatatggtagagccgcagacacgtcgtatgcaggaacgtgctgcggctggctggtgaacttccgatagtgcgggtgttgaatgatttccagttgctaccgattttacatattttttgcatgagagaatttgtaccacctcccaccgaccatctatgactgtacgccactgtccctaggactgctatgtgccggagcggacattacaaacgtccttctcggtgcatgccactgttgccaatgacctgcctaggaattggttagcaagttactaccggattttgtaaaaacagccctcctcatataaaaagtattcgttcacttccgataagcgtcgtaattttctatctttcatcatattctagatccctctgaaaaaatcttccgagtttg

SEQ ID NO: 8 - DNA, 1000 bp, Coliphage lambda
atgtcatatcctataatctggtttttgtttttttgaataataaatgttactgttcttgcggtttggaggaattgattcaaattcaagcgaaataattcagggtcaaaatatgtatcaatgcagcatttgagcaagtgcgataaatctttaagtcttctttcccatggttttttagtcataaaactctccattttgataggttgcatgctagatgctgatatattttagaggtgataaaattaactgcttaactgtcaatgtaatacaagttgtttgatctttgcaatgattcttatcagaaaccatatagtaaattagttacacaggaaatttttaatattattattatcattcattatgtattaaaattagagttgtggcttggctctgctaacacgttgctcataggagatatggtagagccgcagacacgtcgtatgcaggaacgtgctgcggctggctggtgaacttccgatagtgcgggtgttgaatgatttccagttgctaccgattttacatattttttgcatgagagaatttgtaccacctcccaccgaccatctatgactgtacgccactgtccctaggactgctatgtgccggagcggacattacaaacgtccttctcggtgcatgccactgttgccaatgacctgcctaggaattggttagcaagttactaccggattttgtaaaaacagccctcctcatataaaaagtattcgttcacttccgataagcgtcgtaattttctatctttcatcatattctagatccctctgaaaaaatcttccgagtttgctaggcactgatacataactcttttccaataattggggaagtcattcaaatctataataggtttcagatttgcttcaataaattctgactgtagctgctgaaacgttgcggttgaactatatttccttataacttttacgaaagagtttctttgagtaatcacttcactcaagtgcttccctgcctccaaacgatacct